(19) United States
(12) Patent Application Publication
(10) Pub. No.: US 2005/0197558 A1
(43) Pub. Date: Sep. 8, 2005

(54) System and method for performing a virtual endoscopy in a branching structure

(76) Inventors: James P. Williams, Princeton Junction, NJ (US); Bernhard Geiger, Cranbury, NJ (US); Chenyang Xu, Allentown, NJ (US)

Correspondence Address: Siemens Corporation;Intellectual Property Department, 170 Wood Avenue South, Iselin, NJ, 08830, US

(21) Appl. No.: 10/951,188

(22) Filed: Sep. 27, 2004

Related U.S. Application Data
(60) Provisional application No. 60/550,135, filed on Mar. 4, 2004.

(51) Int. Cl. A61B 5/05

(52) U.S. Cl. 600/407

(57) ABSTRACT

A system and method for performing a virtual endoscopy in a branching structure is provided. The method comprises the steps of: determining an initial viewpoint and viewing direction of a virtual endoscope in a branching structure; casting a plurality of rays from the initial viewpoint along the viewing direction; and determining an occurrence of a branch in the branching structure, wherein the occurrence is associated with a cluster that corresponds to the branch.

### CROSS-REFERENCE TO RELATED APPLICATIONS

This application claims the benefit of U.S. Provisional Application No. 60/550,135, filed Mar. 4, 2004, the disclosure of which is herein incorporated by reference.

### BACKGROUND OF THE INVENTION

1. Technical Field

The present invention relates to performing a virtual endoscopy and, more particularly, to performing a virtual endoscopy in a branching structure.

2. Discussion of the Related Art

Virtual endoscopy refers to a method of diagnosis based on computer simulation of standard, minimally invasive endoscopic procedures using patient specific three-dimensional (3D) anatomic data sets. Examples of current endoscopic procedures include bronchoscopy, sinusoscopy, upper gastrointestinal (GI) endoscopy, colonoscopy, cystoscopy, cardioscopy and urethroscopy. Virtual endoscopic visualization of non-invasively obtained patient specific anatomic structures avoids the risks (e.g., perforation, infection, hemorrhage, etc.) associated with real endoscopy and provides the endoscopist with important information prior to performing an actual endoscopic examination. Such understanding can minimize procedural difficulties, decrease patient morbidity, enhance training and foster a better understanding of therapeutic results. In virtual endoscopy, 3D images are created from two-dimensional (2D) computerized tomography (CT) or magnetic resonance (MR) data, for example, by volume rendering. These 3D images are created to simulate images coming from an actual endoscope, e.g., a fiber optic endoscope. This means that a viewpoint of the virtual endoscope has to be chosen inside a lumen of the organ or other human structure, and the rendering of the organ wall has to be done using perspective rendering with a wide angle of view, typically 100 degrees. This viewpoint has to move along the inside of the lumen, which means that a 3D translation and a 3D rotation have to be applied. Controlling these parameters interactively is a challenge.

A commonly used technique for navigating a viewpoint of a virtual endoscope is to plan a “flight” path beforehand and automatically move the viewpoint of the virtual endoscope along this path. This technique, however, has been typically limited to non-branching structures such as pipe networks, tunnel schematics or any other structure in which a volume of space is occupied by a network of closed elongated passages surrounded by a solid or semi-solid border.

Further, biological branching structures typically follow a pattern where the root of the tree is thick and the branches become progressively thinner at each generation. In order to encompass the available volume in a body, the branching structures follow a pattern where the branches tend to occur at acute angles. Because of these inherent geometric characteristics, virtual endoscopic images produced during navigation from terminal branches toward the root are not visualized clearly. Thus, most virtual endoscopic navigational techniques occur from the root to the terminal branches.

Accordingly, there is a need for a technique that enables the “flight path” to be planned so that a virtual endoscope can be automatically moved along this path in a branching structure and that may begin from either the root to the terminal branches or from the terminal branches to the root.

### SUMMARY OF THE INVENTION

The present invention overcomes the foregoing and other problems encountered in the known teachings by providing a system and method for performing a virtual endoscopy in a branching structure.

In one embodiment of the present invention, a method for performing a virtual endoscopy in a branching structure, comprises: determining an initial viewpoint and viewing direction of a virtual endoscope in a branching structure; casting a plurality of rays from the initial viewpoint along the viewing direction; and determining an occurrence of a branch in the branching structure, wherein the occurrence is associated with a cluster that corresponds to the branch.

The method further comprises acquiring three-dimensional (3D) data of the branching structure. The 3D data is acquired by one of a computed tomographic (CT), helical CT, x-ray, positron emission tomographic, fluoroscopic, ultrasound, and magnetic resonance (MR) imaging technique. The method further comprises rendering 3D data of the branching structure. The rendering is performed using one of a raycasting, splatting, shear-warping, texture mapping, surface rendering, and volume rendering technique.

The branching structure is one of a bronchial tree, blood vessel, airway, sinus, and heart. The initial viewpoint and viewing direction are selected by a user. The cluster is also formed by performing a thresholding of a length of the plurality of rays followed by a computation of connected components. The cluster may also be formed by one of a k-means clustering, and mean-shift based clustering of the plurality of rays.

The cluster is further formed by constructing a minimum spanning tree (MST) of endpoints of the plurality of rays and thresholding of an edge length of edges in the MST to separate the endpoints of the plurality of rays. The cluster may also be formed by projecting endpoints of the plurality of rays onto a viewing plane of the virtual endoscope in parallel to form a two-dimensional (2D) image of the endpoints and performing one of a thresholding of a length of the plurality of rays followed by a computation of connected components, k-means clustering, and mean-shift based clustering.

The method further comprises determining a direction to navigate the virtual endoscope by selecting the branch. The selected branch is determined by extracting a longest ray from the cluster. The method further comprises navigating the virtual endoscope from the viewpoint to the selected branch. The navigation is one of a “top-down” and “bottom-up” type navigation. The method further comprises storing the occurrence of the branch.

In another embodiment of the present invention, a method for performing a virtual endoscopy in a branching structure, comprises: determining an initial viewpoint and viewing direction of a virtual endoscope in a branching structure; selecting a preferred direction of the virtual endoscope; casting a plurality of rays from the initial viewpoint; determining a longest ray from the initial viewpoint using the preferred direction as a weight; and navigating through the branching structure to the preferred direction.

The method further comprises acquiring 3D data of the branching structure. The 3D data is acquired by one of a CT, helical CT, x-ray, positron emission tomographic, fluoroscopic, ultrasound, and MR imaging technique. The method further comprises rendering the 3D data of the branching structure. The rendering is performed using one of a raycasting, splatting, shear-warping, texture mapping, surface rendering, and volume rendering technique.

The branching structure is one of a bronchial tree, blood vessel, airway, sinus, and heart. The preferred direction is selected by a user. The weight is determined by calculating an inner product of the preferred direction and each of the plurality of rays.

In yet another embodiment of the present invention, a system for performing a virtual endoscopy in a branching structure, comprises: a memory device for storing a program; a processor in communication with the memory device, the processor operative with the program to: determine an initial viewpoint and viewing direction of a virtual endoscope in a branching structure; cast a plurality of rays from the initial viewpoint along the viewing direction using a raycasting technique; and determine a location of a branch in the branching structure, wherein the location is associated with a cluster that corresponds to the branch. The processor is further operative with the program code to render 3D data of the branching structure. The cluster is formed by performing a thresholding of a length of the plurality of rays followed by a computation of connected components.

In another embodiment of the present invention, a system for performing a virtual endoscopy in a branching structure, comprises: a memory device for storing a program; a processor in communication with the memory device, the processor operative with the program to: determine an initial viewpoint and viewing direction of a virtual endoscope in a branching structure; select a preferred direction of the virtual endoscope; cast a plurality of rays from the initial viewpoint using a raycasting technique; determine a longest ray from the initial viewpoint using the preferred direction as a weight; and navigate through the branching structure to the preferred direction. The weight is determined by calculating an inner product of the preferred direction and each of the plurality of rays.

In yet another embodiment of the present invention, a computer program product comprising a computer useable medium having computer program logic recorded thereon for performing a virtual endoscopy, the computer program logic comprises: program code for determining an initial viewpoint and viewing direction of a virtual endoscope in a branching structure; program code for casting a plurality of rays from the initial viewpoint along the viewing direction; and program code for determining an occurrence of a branch in the branching structure, wherein the occurrence is associated with a cluster that corresponds to the branch.

In another embodiment of the present invention, a computer program product comprising a computer useable medium having computer program logic recorded thereon for performing a virtual endoscopy, the computer program logic comprises: program code for determining an initial viewpoint and viewing direction of a virtual endoscope in a branching structure; program code for selecting a preferred direction of the virtual endoscope; program code for casting a plurality of rays from the initial viewpoint; program code for determining a longest ray from the initial viewpoint using the preferred direction as a weight, wherein the weight is determined by calculating an inner product of the preferred direction and each of the plurality of rays; and program code for navigating through the branching structure to the preferred direction.

In yet another embodiment of the present invention, a system for performing a virtual endoscopy in a branching structure, comprises: means for determining an initial viewpoint and viewing direction of a virtual endoscope in a branching structure; means for casting a plurality of rays from the initial viewpoint along the viewing direction; and means for determining an occurrence of a branch in the branching structure, wherein the occurrence is associated with a cluster that corresponds to the branch.

In another embodiment of the present invention, a system for performing a virtual endoscopy in a branching structure, comprises: means for determining an initial viewpoint and viewing direction of a virtual endoscope in a branching structure; means for selecting a preferred direction of the virtual endoscope; means for casting a plurality of rays from the initial viewpoint; means for determining a longest ray from the initial viewpoint using the preferred direction as a weight; and means for navigating through the branching structure to the preferred direction.

The foregoing features are of representative embodiments and are presented to assist in understanding the invention. It should be understood that they are not intended to be considered limitations on the invention as defined by the claims, or limitations on equivalents to the claims. Therefore, this summary of features should not be considered dispositive in determining equivalents. Additional features of the invention will become apparent in the following description, from the drawings and from the claims.

### BRIEF DESCRIPTION OF THE DRAWINGS

FIG. 1 is a block diagram of a system for performing a virtual endoscopy in a branching structure according to an exemplary embodiment of the present invention;

FIG. 2 is a flowchart showing an operation of a method for performing a virtual endoscopy in a branching structure according to an exemplary embodiment of the present invention;

FIG. 3 illustrates a clustering of rays in a branching structure according to an exemplary embodiment of the present invention;

FIG. 4 is a flowchart showing an operation of a method for performing a virtual endoscopy in a branching structure according to another exemplary embodiment of the present invention; and

FIG. 5 illustrates a reverse virtual endoscopy in a branching structure according to an exemplary embodiment of the present invention.

### DETAILED DESCRIPTION OF EXEMPLARY EMBODIMENTS

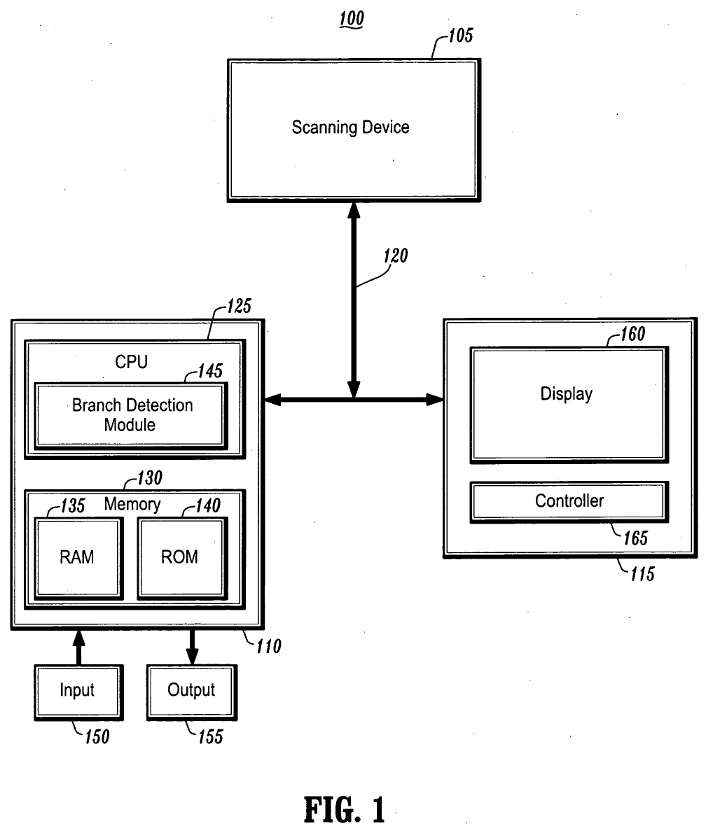

FIG. 1 is a block diagram of a system 100 for performing a virtual endoscopy in a branching structure according to an exemplary embodiment of the present invention. As shown in FIG. 1, the system 100 includes, inter alia, a scanning device 105, a personal computer (PC) 110 and an operator's console and/or virtual navigation terminal 115 connected over, for example, an Ethernet network 120. The scanning device 105 may be a magnetic resonance imaging (MRI) device, a computed tomography (CT) imaging device, a helical CT device, a positron emission tomography (PET) device, a two-dimensional (2D) or three-dimensional (3D) fluoroscopic imaging device, a 2D, 3D, or four-dimensional (4D) ultrasound imaging device, or an x-ray device, etc.

The PC 110, which may be a portable or laptop computer, a personal digital assistant (PDA), etc., includes a central processing unit (CPU) 125 and a memory 130, which are connected to an input 150 and an output 155. The CPU 125 includes a branch detection module 145 that includes one or more methods for determining a location of a branch in a medical image of a branching structure such as a bronchial tree or blood vessel. The CPU 125 may also include a protrusion detection module, which is a computer-aided detection (CAD) module for detecting protrusions such as polyps in a medical image, and a diagnostic module, which is used to perform automated diagnostic or evaluation functions of medical image data.

The memory 130 includes a random access memory (RAM) 135 and a read only memory (ROM) 140. The memory 130 can also include a database, disk drive, tape drive, etc., or a combination thereof. The RAM 135 functions as a data memory that stores data used during execution of a program in the CPU 125 and is used as a work area. The ROM 140 functions as a program memory for storing a program executed in the CPU 125. The input 150 is constituted by a keyboard, mouse, etc., and the output 155 is constituted by a liquid crystal display (LCD), cathode ray tube (CRT) display, printer, etc.

The operation of the system 100 is controlled from the virtual navigation terminal 115, which includes a controller 165, for example, a keyboard, and a display 160, for example, a CRT display. The virtual navigation terminal 115 communicates with the PC 110 and the scanning device 105 so that 2D image data collected by the scanning device 105 can be rendered into 3D data by the PC 110 and viewed on the display 160. It is to be understood that the PC 110 can be configured to operate and display information provided by the scanning device 105 absent the virtual navigation terminal 115, using, for example, the input 150 and output 155 devices to execute certain tasks performed by the controller 165 and display 160.

The virtual navigation terminal 115 further includes any suitable image rendering system/tool/application that can process digital image data of an acquired image dataset (or portion thereof) to generate and display 2D and/or 3D images on the display 160. More specifically, the image rendering system may be an application that provides 2D/3D rendering and visualization of medical image data, and which executes on a general purpose or specific computer workstation. Moreover, the image rendering system enables a user to navigate through a 3D image or a plurality of 2D image slices. The PC 110 may also include an image rendering system/tool/application for processing digital image data of an acquired image dataset to generate and display 2D and/or 3D images.

As shown in FIG. 1, the branch detection module 145 may also be used by the PC 110 to receive and process digital medical image data, which as noted above, may be in the form of raw image data, 2D reconstructed data (e.g., axial slices), or 3D reconstructed data such as volumetric image data or multiplanar reformats, or any combination of such formats. The data processing results can be output from the PC 110 via the network 120 to an image rendering system in the virtual navigation terminal 115 for generating 2D and/or 3D renderings of image data in accordance with the data processing results, such as segmentation of organs or anatomical structures, color or intensity variations, and so forth.

It is to be understood that CAD systems and methods according to the present invention for performing a virtual endoscopy in a branching structure may be implemented as extensions or alternatives to conventional CAD methods or other automated visualization and detection methods for processing image data. Further, it is to be appreciated that the exemplary systems and methods described herein can be readily implemented with 3D medical images and CAD systems or applications that are adapted for a wide range of imaging modalities (e.g., CT, MRI, etc.) and for diagnosing and evaluating various abnormal anatomical structures or lesions such as colonic polyps, aneurysms, lung nodules, etc. In this regard, although exemplary embodiments may be described herein with reference to particular imaging modalities or particular anatomical features, nothing should be construed as limiting the scope of the invention.

It is to be further understood that the present invention may be implemented in various forms of hardware, software, firmware, special purpose processors, or a combination thereof. In one embodiment, the present invention may be implemented in software as an application program tangibly embodied on a program storage device (e.g., magnetic floppy disk, RAM, CD ROM, DVD, ROM, and flash memory). The application program may be uploaded to, and executed by, a machine comprising any suitable architecture.

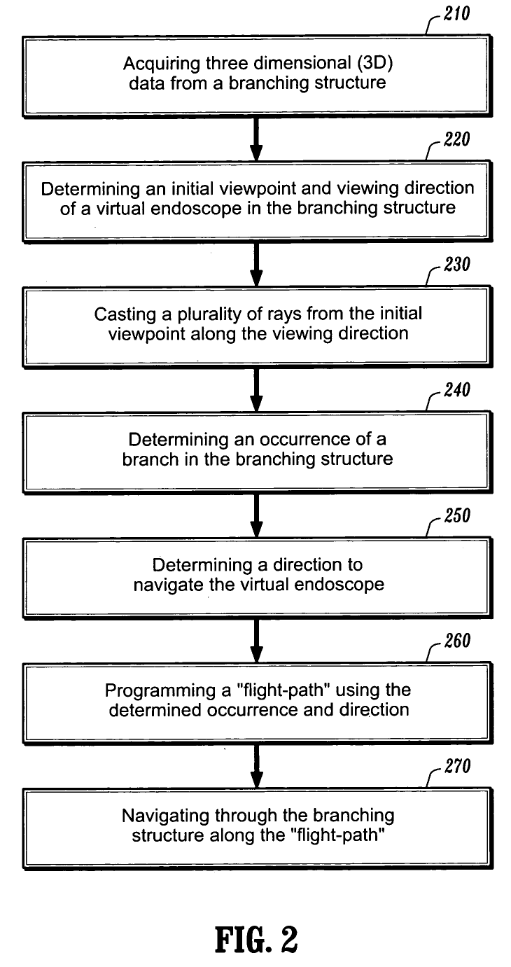

FIG. 2 is a flowchart showing an operation of a method for performing a virtual endoscopy in a branching structure according to an exemplary embodiment of the present invention. As shown in FIG. 2, 3D data is acquired from a branching structure, which in this example is a bronchial tree (step 210). This is accomplished by using the scanning device 105, in this example a CT scanner, which is operated at the operator's console 115, to scan the bronchial tree thereby generating a series of 2D images associated with the bronchial tree. The 2D images of the bronchial tree are then converted or transformed into a 3D rendered image. It is to be understood that the branching structure can be in addition to the bronchial tree any one of a blood vessel, airway, sinus, heart, etc.

After the 3D data is acquired from the bronchial tree, an initial viewpoint and viewing direction of a virtual endoscope in the branching structure are determined (step 220). The initial viewpoint and viewing direction may be determined interactively by a user using, for example, a mouse, or automatically by a conventional method for determining a starting position for virtual endoscopic navigation. Step 220 is accomplished by using data associated with a rendering of the 3D data using a conventional rendering technique such as raycasting, splatting, shear-warping, 3D texture mapping, surface rendering, volume rendering etc. For purposes of this example, a raycasting technique is employed.

After step 220, a plurality of rays are cast from the initial point of the virtual endoscope in the branching structure (step 230). It is to be understood, however, that the rays are cast regardless of the rendering technique employed. However, when using the raycasting technique in step 220, the rays are cast and step 230 is not necessary. These rays produce a depth value for every pixel in the 3D data. The resulting depth values will form a cluster of pixels around the longest ray or rays which point “forward” down the bronchial tree. The resulting clusters each correspond to a branch in the branching structure, thus enabling an occurrence of a branch to be detected (step 240).

In order to determine the clusters, a thresholding of the ray length followed by a computation of connected components is performed. A threshold for the distance can also be selected interactively by the user, or a preset for the given application (e.g., airways, large blood vessels, small blood vessels) may be used, or the threshold can be calculated automatically depending on the current diameter of the vessel where the viewpoint is located (here the rays can be used to estimate the diameter of the vessel).

In more detail, when using the raycasting technique (as discussed for steps 220 or 230), a ray is cast through each pixel in an image plane made up of the 3D data. For each ray that is longer than a given threshold, the corresponding pixel is colored, for example, white. All other pixels are set to black. To find the connected components, all of the pixels in the image plane are searched. Once a first white pixel is found, it is set as the seed for a first cluster. Then all neighboring pixels are observed. If they are also white, they will be added to the first cluster. This process will be repeated with the neighboring pixels until all pixels that are connected to each other have been added and the first cluster stops growing.

Next, all of the remaining pixels are searched until another white pixel that is not part of the first cluster is found. This will be the seed for a second cluster. The process described for the first cluster is then repeated for the second cluster until the second cluster stops growing. It is to be understood that the above processes will continue until all of the pixels in the in the image plane made up of the 3D data have been processed.

It should also be understood that in order for the rays and/or their endpoints to belong to the same cluster, they must also be mutually visible to one another. That is, given two endpoints, for example, A and B, A can be in the same cluster as B if a line segment connecting A and B does not pass through any opaque structures such as a bronchial wall. Thus, if all endpoints [A1 . . . AK] belong to the same cluster, then for all i,j (i.e., endpoints) such that 1<=i, j<=K, Ai and Aj are mutually visible.

Once the branches are detected, a longest ray from each cluster can be extracted to provide a choice of directions for continued progress of the virtual endoscope through the bronchial tree (step 250). In other words, a direction to navigate the virtual endoscope is determined by choosing which branch to navigate. Thus, once the cluster has been identified, for example, the first cluster, you select the longest ray of all the rays in that cluster as the direction of, for example, a first branch, and from the second cluster, you select the longest ray as the direction of, for example, the second branch.

At this point, the determined directions for navigating the virtual endoscope may be used to augment an existing or used to create a “flight-path” plan and/or program for navigating through the bronchial tree or any other branching structure (step 260). Prior to generating the “flight-path” program, the data associated with the directions for navigating the virtual endoscope can be stored, for example, in the memory 130 of the CPU 125 for further manipulation and/or analysis. Once the “flight-path” has been programmed, a medical expert can navigate through the bronchial tree along the “flight-path” (step 270). In other words, the operator of the virtual navigation terminal 115 performs a planned or guided navigation according to the “flight-path” of the virtual organ being examined.

It is to be understood that although steps 260 and 270 are shown in this example, a user may begin navigating through the branching structure immediately after step 240. Thus, steps 220-240 may be continuously repeated when a user navigates through the branching structure. During their navigation, the user may be informed of the presence of an event such as a branch point or the location of two or more branches. This enables the user to make a decision as to where they wish to maneuver the virtual endoscope.

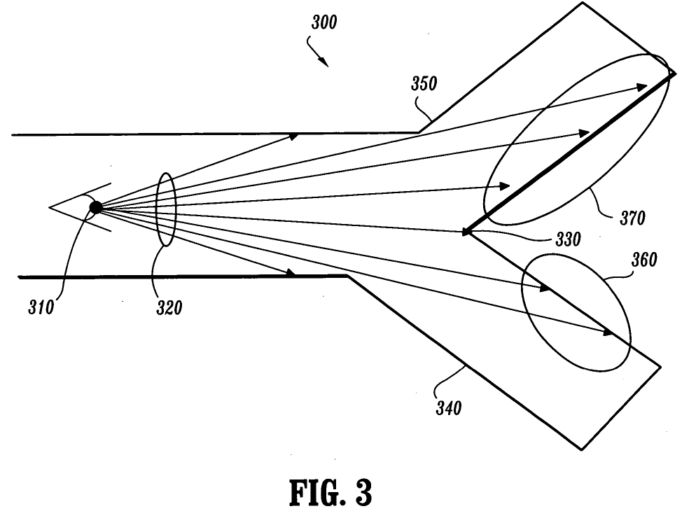

FIG. 3 is provided to illustrate the clustering of rays in a branching structure 300 in accordance with an exemplary embodiment of the present invention. As shown in FIG. 3, after the image is rendered an initial viewpoint 310 is determined and a plurality of rays 320 are cast from the initial viewpoint 310 using the raycasting technique discussed above. As illustrated, some of the rays do not travel very far and immediately strike a surface of the branching structure 300 before reaching a branch point 330. However, some of the rays travel beyond the branch point 330 into branches 340, 350. These rays cluster together and form clusters 360, 370, which are used to determine a direction of a virtual endoscope by, for example, extracting the longest ray from each cluster to provide a choice of directions for progressing the virtual endoscope through the branching structure 300 as discussed above with respect to FIG. 2.

It is to be understood that additional conventional clustering techniques may be used in accordance with the present invention. These techniques may be, for example, k-means clustering, and mean-shift based clustering, both of which can be used to extract groups and/or clusters based on their proximity to the endpoints of rays. In addition, a minimum spanning tree (MST) can be constructed using the ray endpoints and a thresholding of an edge length of edges in the MST can be used to separate the endpoints of the rays to identify clusters and thus locate branches. Further, the ray endpoints can be projected onto a viewing plane of the virtual endoscope in parallel to form a 2D image of the endpoints. This projection can then be clustered using one of the above-described clustering methods to locate the branches.

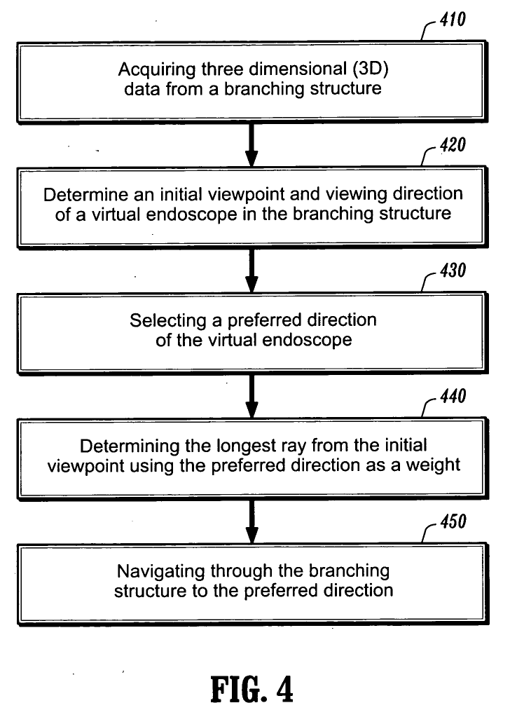

FIG. 4 is a flowchart showing an operation of a method for performing a virtual endoscopy in a branching structure according to another exemplary embodiment of the present invention. As shown in FIG. 4, 3D data is acquired from a branching structure such as a bronchial tree using a CT scanner (step 410). After the 3D data is acquired, it is rendered using a raycasting technique and an initial viewpoint and direction associated with the viewpoint can be determined (step 420). This may be accomplished by clicking on a mouse to select an initial viewing location in the 3D data facing in a direction for which virtual endoscopic navigation is desired. Once the initial viewpoint and direction are selected, a user selects a preferred direction for which virtual navigation is desired (step 430). This may also be accomplished by clicking on a mouse and selecting a preferred direction in the 3D data.

Next, rays are cast from the initial viewpoint to the preferred direction and the longest rays are determined (step 440). In this step, once the longest rays are determined they are combined with the preferred direction and used to modify the endoscope path. In order to determine the longest rays and combine them with the preferred direction, the longest rays must form clusters that are separable in a 2-parameter space. The rays are then parameterized by two angles (ρ,θ. Thus, a ray aligned with a vector associated with the initial viewpoint will have (ρ,θ)=(0,0). As rays that are cast diverge horizontally from the vector associated with the initial viewpoint, the magnitude of ρ increases, and as rays that are cast diverge vertically from the vector associated with the initial viewpoint, the magnitude of θ increases. The resulting (ρ,θ) value is thus associated with the preferred direction.

It is to be understood that the ray length determined in step 440 is not chosen in the standard Euclidean sense. Instead, all of the rays cast are taken and scaled relative to the user's selected preferred direction. This is accomplished in the following manner.

First, a view vector is denoted as V, where V is a unit vector associated with the preferred direction. The user's position, for example, a position of their mouse, is denoted as P=(px,py). Next, the position P is converted to a preferred direction vector D, where D is a vector created by taking a line segment connecting the viewer's position with the position P and then normalizing this segment.

For each ray cast from the viewer, referred here to as Ri, a scaled version of the rays S(Ri) is calculated. The scaling function may be of the form: S(Ri)=g(<Ri,D>)*Ri, where <Ri,D> denotes the inner product of the vector. The inner product is equal to the cosine of the angle formed by the vectors Ri & D. Thus, when Ri and D are equal the value of the inner product is +1, and when Ri and D are orthogonal, the inner product is 0. It is to be understood however that the function g(<Ri,D>)*Ri can take many forms. It could be, for example, the identity function, i.e., g(<Ri,D>)=<Ri,d>.

It is to be further understood that many additional parameters can be used to tune the behavior of the function g, because, for example, g enables the preferred direction to become stronger or weaker thus pulling the endoscope in a direction more strongly or weakly. Parameters that can be used to modulate the sensitivity of the inner product of g can be, for example, polynomial, exponential, logarithmic, trigonometric functions, etc. In addition, velocity and inertia simulations of the moving viewpoint can be incorporated to increase the sensitivity of the inner product value of g when the user specifies a low velocity motion or to decrease sensitivity to directional input when the user is navigating at a high velocity.

Further, data collected from user interactions prior to a current navigation session can be used to either increase or decrease the sensitivity of the inner product value of g. For example, one can assume that the user's past reactions to the navigation algorithm for choosing a branch will also reflect their future preferences. Thus, one can compute a set of features from a current ray distribution and evaluate the apparent significance of these features to past decisions that the user has made to deviate from the current main path using machine learning algorithms. This data can then be used to automatically weigh the user's navigation preference towards a detected branch.

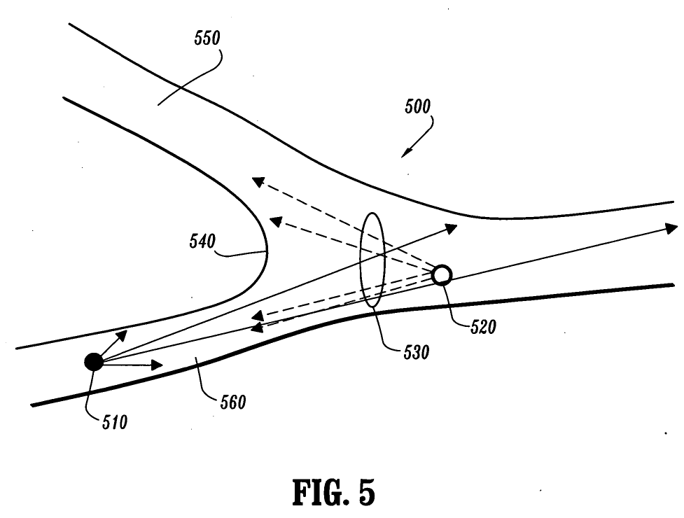

The above discussion with regard to FIGS. 1-4 focused primarily on “top-down” exploration of branching structures. That is, from a root to a terminal branch of the branching structure. FIG. 5, however, illustrates a reverse “bottom-up” virtual endoscopy in a branching structure in accordance with an exemplary embodiment of the present invention. That is, from a terminal branch to a root of the branching structure.

Bottom-up virtual endoscopy is accomplished, for example, by moving a viewing position 510 from the bottom of a branching structure 500. A look-ahead position 520 is specified as a certain distance from the viewing position 510 along the longest ray cast from the viewing position 510. In other words, the look-ahead position 520 is a point that is being dragged by the user while they are moving through a virtual model of the branching structure. From the look-ahead position 520, one or more rays 530 are cast in an inverse direction using the techniques described above with reference to FIGS. 1-4. Once a branch point 540 is detected, a desired branch 550, 560 may be selected and reverse navigation may continue throughout the branching structure 500.

It is to be understood that there are a variety of modes in which a user can deliver navigation control or in which information regarding branch detection and prior navigation decisions can be displayed when performing a virtual endoscopy in a branching structure according to the present invention. For example, when employing an “exhaustive navigation” mode, all branches that have been detected may be explored either in a pre-processing step or online as they are discovered during the process of navigation. One option for displaying this information is to take all of the branches and split the visualization into multiple windows. In the alternative, a visual index into all of the potential paths can be created and overlaid onto an external or schematic view of the structure in which the user is navigating.

In another mode, referred to as “detect and choose”, when a branch is automatically detected the user may choose which branch to pursue using an input device. As in exhaustive navigation, a visual display of the explored region and the branches that have been detected can be created. Further, in a point-to-point navigation mode, a user may provide a start point inside the structure where navigation is to begin (optionally this start point could be chosen automatically by an algorithm) and an end point also inside the structure. The system will then perform a search starting at one or both points for a navigation route which connects the two endpoints. Branch detection as described above is used during this search to compute possible paths of exploration.

It is to be understood that because some of the constituent system components and method steps depicted in the accompanying figures may be implemented in software, the actual connections between the system components (or the process steps) may differ depending on the manner in which the present invention is programmed. Given the teachings of the present invention provided herein, one of ordinary skill in the art will be able to contemplate these and similar implementations or configurations of the present invention.

It should also be understood that the above description is only representative of illustrative embodiments. For the convenience of the reader, the above description has focused on a representative sample of possible embodiments, a sample that is illustrative of the principles of the invention. The description has not attempted to exhaustively enumerate all possible variations. That alternative embodiments may not have been presented for a specific portion of the invention, or that further undescribed alternatives may be available for a portion, is not to be considered a disclaimer of those alternate embodiments. Other applications and embodiments can be straightforwardly implemented without departing from the spirit and scope of the present invention. It is therefore intended, that the invention not be limited to the specifically described embodiments, because numerous permutations and combinations of the above and implementations involving non-inventive substitutions for the above can be created, but the invention is to be defined in accordance with the claims that follow. It can be appreciated that many of those undescribed embodiments are within the literal scope of the following claims, and that others are equivalent.

1. A method for performing a virtual endoscopy in a branching structure, comprising:
determining an initial viewpoint and viewing direction of a virtual endoscope in a branching structure;
casting a plurality of rays from the initial viewpoint along the viewing direction; and
determining an occurrence of a branch in the branching structure, wherein the occurrence is associated with a cluster that corresponds to the branch.

2. The method of claim 1, further comprising:
acquiring three-dimensional (3D) data of the branching structure.

3. The method of claim 2, wherein the 3D data is acquired by one of a computed tomographic (CT), helical CT, x-ray, positron emission tomographic, fluoroscopic, ultrasound, and magnetic resonance (MR) imaging technique.

4. The method of claim 1, further comprising:
rendering 3D data of the branching structure.

5. The method of claim 4, wherein the rendering is performed using one of a raycasting, splatting, shear-warping, texture mapping, surface rendering, and volume rendering technique.

6. The method of claim 1, wherein the branching structure is one of a bronchial tree, blood vessel, airway, sinus, and heart.

7. The method of claim 1, wherein the initial viewpoint and viewing direction are selected by a user.

8. The method of claim 1, wherein the cluster is formed by performing a thresholding of a length of the plurality of rays followed by a computation of connected components.

9. The method of claim 1, wherein the cluster is formed by one of a k-means clustering, and mean-shift based clustering of the plurality of rays.

10. The method of claim 1, wherein the cluster is formed by constructing a minimum spanning tree (MST) of endpoints of the plurality of rays and thresholding of an edge length of edges in the MST to separate the endpoints of the plurality of rays.

11. The method of claim 1, wherein the cluster is formed by projecting endpoints of the plurality of rays onto a viewing plane of the virtual endoscope in parallel to form a two-dimensional (2D) image of the endpoints and performing one of a thresholding of a length of the plurality of rays followed by a computation of connected components, k-means clustering, and mean-shift based clustering.

12. The method of claim 1, further comprising:
determining a direction to navigate the virtual endoscope by selecting the branch.

13. The method of claim 12, wherein the selected branch is determined by extracting a longest ray from the cluster.

14. The method of claim 12, further comprising:
navigating the virtual endoscope from the viewpoint to the selected branch.

15. The method of claim 14, wherein the navigation is one of a “top-down” and “bottom-up” type navigation.

16. The method of claim 1, further comprising:
storing the occurrence of the branch.

17. A method for performing a virtual endoscopy in a branching structure, comprising:
determining an initial viewpoint and viewing direction of a virtual endoscope in a branching structure;
selecting a preferred direction of the virtual endoscope;
casting a plurality of rays from the initial viewpoint;
determining a longest ray from the initial viewpoint using the preferred direction as a weight; and
navigating through the branching structure to the preferred direction.

18. The method of claim 17, further comprising:
acquiring three-dimensional (3D) data of the branching structure.

19. The method of claim 18, wherein the 3D data is acquired by one of a computed tomographic (CT), helical CT, x-ray, positron emission tomographic, fluoroscopic, ultrasound, and magnetic resonance (MR) imaging technique.

20. The method of claim 17, further comprising:
rendering 3D data of the branching structure.

21. The method of claim 20, wherein the rendering is performed using one of a raycasting, splatting, shear-warping, texture mapping, surface rendering, and volume rendering technique.

22. The method of claim 17, wherein the branching structure is one of a bronchial tree, blood vessel, airway, sinus, and heart.

23. The method of claim 17, wherein the preferred direction is selected by a user.

24. The method of claim 17, wherein the weight is determined by calculating an inner product of the preferred direction and each of the plurality of rays.

25. A system for performing a virtual endoscopy in a branching structure, comprising:
a memory device for storing a program;
a processor in communication with the memory device, the processor operative with the program to:
determine an initial viewpoint and viewing direction of a virtual endoscope in a branching structure;
cast a plurality of rays from the initial viewpoint along the viewing direction using a raycasting technique; and
determine a location of a branch in the branching structure, wherein the location is associated with a cluster that corresponds to the branch.

26. The system of claim 25, wherein the processor is further operative with the program code to:
render 3D data of the branching structure.

27. The system of claim 25, wherein the cluster is formed by performing a thresholding of a length of the plurality of rays followed by a computation of connected components.

28. A system for performing a virtual endoscopy in a branching structure, comprising:
a memory device for storing a program;
a processor in communication with the memory device, the processor operative with the program to:
determine an initial viewpoint and viewing direction of a virtual endoscope in a branching structure;
select a preferred direction of the virtual endoscope;
cast a plurality of rays from the initial viewpoint using a raycasting technique;
determine a longest ray from the initial viewpoint using the preferred direction as a weight; and
navigate through the branching structure to the preferred direction.

29. The system of claim 28, wherein the weight is determined by calculating an inner product of the preferred direction and each of the plurality of rays.

30. A computer program product comprising a computer useable medium having computer program logic recorded thereon for performing a virtual endoscopy, the computer program logic comprising:
program code for determining an initial viewpoint and viewing direction of a virtual endoscope in a branching structure;
program code for casting a plurality of rays from the initial viewpoint along the viewing direction; and
program code for determining an occurrence of a branch in the branching structure, wherein the occurrence is associated with a cluster that corresponds to the branch.

31. A computer program product comprising a computer useable medium having computer program logic recorded thereon for performing a virtual endoscopy, the computer program logic comprising:
program code for determining an initial viewpoint and viewing direction of a virtual endoscope in a branching structure;
program code for selecting a preferred direction of the virtual endoscope;
program code for casting a plurality of rays from the initial viewpoint;
program code for determining a longest ray from the initial viewpoint using the preferred direction as a weight, wherein the weight is determined by calculating an inner product of the preferred direction and each of the plurality of rays; and
program code for navigating through the branching structure to the preferred direction.

32. A system for performing a virtual endoscopy in a branching structure, comprising:
means for determining an initial viewpoint and viewing direction of a virtual endoscope in a branching structure;
means for casting a plurality of rays from the initial viewpoint along the viewing direction; and
means for determining an occurrence of a branch in the branching structure, wherein the occurrence is associated with a cluster that corresponds to the branch.

33. A system for performing a virtual endoscopy in a branching structure, comprising:
means for determining an initial viewpoint and viewing direction of a virtual endoscope in a branching structure;
means for selecting a preferred direction of the virtual endoscope;
means for casting a plurality of rays from the initial viewpoint;
means for determining a longest ray from the initial viewpoint using the preferred direction as a weight; and
means for navigating through the branching structure to the preferred direction.
